(19) United States
(12) Patent Application Publication
(10) Pub. No.: US 2011/0229922 A1
(43) Pub. Date: Sep. 22, 2011

(54) SCREENING ASSAYS FOR THE IDENTIFICATION OF BACE2 INHIBITORS

(76) Inventors: Jeremy Beauchamp, Riehen (GB); Heinz Doebeli, Ziefen (CH); Hugues Matile, Basel (CH); Cristiano Migliorini, Genf (CH); Michael Prummer, Zuerich (CH); Paolo Salvioni, Pully (CH); Haiyan Wang, Allschwil (CH)

(21) Appl. No.: 13/131,331

(22) PCT Filed: Nov. 26, 2009
(86) PCT No.: PCT/EP09/65918

§ 371 Date: May 26, 2011

(30) Foreign Application Priority Data
Dec. 2, 2008 (EP) 08020867.1

(51) Int. Cl. C12Q 1/37

(52) U.S. Cl. 435/23

(57) ABSTRACT

The present invention relates to screening assays for the identification of BACE2 inhibitors.

The present invention relates to screening assays for the identification of BACE2 inhibitors. These compounds can be used for the treatment of metabolic disorders.

Defective glucose-stimulated insulin secretion and decreased β-cell mass are the main causes of hyperglycemia in type 2 diabetes mellitus. The transmembrane protein Tmem27 (Collectrin) is expressed in pancreatic β-cells where it regulates pancreatic β-cell mass, and insulin secretion. Tmem27 is inactivated at the plasma membrane by proteolytic cleavage and shedding.

Angiotensin converting enzyme 2 (ACE2) is a multidomain membrane protein with physiological roles including the cleavage of Angiotensin II to Angiotensin. Thus increased ACE2 activity has the potential to give protection against metabolic diseases including hypertension. (Ingelfinger “Angiotensin-converting enzyme 2: implications for blood pressure and kidney disease.” Curr Opin Nephrol Hypertens. (2009) 18(1):79-84.) In the pancreas, reduced ACE2 is associated with impared glucose homeostasis (Niu et al. “Loss of angiotensin-converting enzyme 2 leads to impaired glucose homeostasis in mice.” Endocrine. (2008), 34(1-3):56-61.) therefore preservation of ACE2 may also have beneficial effects in diabetes. ACE2 and TMEM27 have close sequence homology in their extracellular domains and it is therefore possible that they share the same shedding protease.

BACE1 is a β-secretase (β-site of APP cleaving enzyme), belongs to the class of aspartic acid proteases and has been implicated in the pathogenesis of Alzheimer disease and in the formation of myelin sheaths in peripheral nerve cells. It is a transmembrane protein, contains two active site aspartate residues in its extracellular protein domain and may function as a dimer.

The generation of the 40 or 42 amino acid long amyloid β-proteins that aggregate in the brain of Alzheimer's patients requires two sequential cleavages of the amyloid precursor protein (APP). Extracellular cleavage of APP by BACE1 releases a soluble extracellular fragment and is followed by APP cleavage within its transmembrane domain by γ-secretase (presenilin). The second cleavage releases the intracellular domain of APP and amyloid-β. Since α-secretase cleaves APP closer to the cell membrane than BACE1 does, it removes a fragment of the amyloid-β peptide. Initial cleavage of APP by a α-secretase rather than BACE1 prevents eventual generation of amyloid-β.

Unlike APP and the presenilin proteins important in γ-secretase, no known mutations in the gene encoding BACE1 cause the early-onset, familial Alzheimer's disease. However, levels of this enzyme have been shown to be elevated in Alzheimer's. The physiological purpose of BACE1's cleavage of APP and other transmembrane proteins is unknown. BACE2 is a close homolog of BACE1.

The aim of the present invention was to provide new assays for the identification of compounds for the treatment of metabolic disorders.

The present invention is based on the finding that BACE2 is the protease cleaving Tmem27. Inhibition of BACE2 leads to inhibition of Tmem27 shedding and an increase of the full length protein. In cells whose proliferation is dependent on the presence of full length Tmem27 protein, inhibition of BACE2 mediated Tmem27 cleavage leads to an increase of cell proliferation.

In a first aspect, the present invention provides a method for identifying a BACE2 inhibitor comprising: providing a cell expressing a Tmem27 polypeptide wherein proliferation of the cell is dependent on BACE2 mediated Tmem27 cleavage, contacting a mixture comprising a BACE2 polypeptide and the cell expressing the Tmem27 polypeptide with a candidate compound and determining whether the candidate compound modulates cell proliferation, wherein an increase of cell proliferation is indicative for a BACE2 inhibitor.

In a preferred embodiment, the cell expresses Tmem27 polypeptide and BACE2.

In a further preferred embodiment, the cell is a beta cell line, preferably a MIN6 B1 or INS-1e cell line.

In yet another preferred embodiment, cell proliferation is measured by confocal microscopy.

In a second aspect, the present invention provides a method for identifying a BACE2 inhibitor comprising: contacting BACE2 and a peptide comprising a Tmem27 derived BACE2 cleavage site or an ACE2 derived BACE2 cleavage site with a candidate compound and determining cleavage of the peptide.

In a preferred embodiment, the Tmem27 derived peptide comprises a peptide having the sequence set forth in Seq. Id. No. 1 (QTLEFLKIPS).

In a further preferred embodiment, the ACE2 derived peptide comprises a peptide having the sequence set forth in Seq. Id. No. 14 (NSLEFLGIQP).

In a further preferred embodiment, cleavage of the Tmem27 derived peptide or the ACE2 derived peptide is determined in a fluorophore fluorescence resonance energy transfer (FRET) assay.

In yet another preferred embodiment, the Tmem27 derived peptide or the ACE2 derived peptide is labelled with dabsyl at the N-terminus and a fluorescent dye at the C-terminus.

In a further preferred embodiment, the fluorescent dye is Lucifer yellow.

In a further preferred embodiment, cleavage of the Tmem27 derived peptide or the ACE2 derived peptide is determined in a fluoresence quench assay.

In yet another preferred embodiment, the Tmem27 derived peptide or the ACE2 derived peptide is labelled with MR121 at the N-terminus and tryptophan at the C-terminus.

In another aspect, the present invention relates to the use of an ACE2 peptide for the identification of a BACE2 inhibitor.

In a further aspect, the present invention provides a peptide selected from the group consisting of Seq. Id. No. 1 and Seq. Id. No. 5-18.

The BACE2 used in the methods of the present invention can be isolated from cell membranes obtained from cells expressing BACE2 or can be isolated from cells expressing BACE2. Alternatively, BACE2 may be partially or fully synthesized by traditional chemical synthesis and/or recombinant DNA technology.

The term “Tmem27” is used herein to refer to native Tmem27 sequence from any animal, e.g. mammalian, species, including humans, and Tmem27 variants. The Tmem27 polypeptides may be isolated from a variety of sources, including human tissue types or prepared by recombinant and/or synthetic methods.

“Native sequence Tmem27” refers to a polypeptide having the same amino acid sequence as a Tmem27 polypeptide occurring in nature regardless of its mode of preparation. A native sequence Tmem27 may be isolated from nature, or prepared by recombinant and/or synthetic methods. The term “native sequence Tmem27” specifically encompasses naturally occurring truncated or secreted forms, naturally occurring variant forms (e.g. alternatively spliced forms), and naturally occurring allelic variants of Tmem27. The identifier of the human Tmem27 polypeptide in the swissprot database is Q9HBJ8 (Seq. Id. No. 2).

The term “Tmem27 variant” refers to amino acid sequence variants of a native sequence Tmem27 containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence Tmem27. The term “Tmem27 variant” refers as well to Tmem27 fragments which can be processed by BACE2 e.g. truncated Tmem27 polypeptides which are still a substrate for BACE2.

The term “BACE2” is used herein to refer to native sequence BACE2 from any animal, e.g. mammalian, species, including humans, and BACE2 variants (which are further defined below). The BACE2 polypeptides may be isolated from a variety of sources, including human tissue types or prepared by recombinant and/or synthetic methods.

“Native sequence BACE2” refers to a polypeptide having the same amino acid sequence as a BACE2 polypeptide occurring in nature regardless of its mode of preparation. A native sequence BACE2 may be isolated from nature, or prepared by recombinant and/or synthetic methods. The term “native sequence BACE2” specifically encompasses naturally occurring truncated or secreted forms, naturally occurring variant forms (e.g. alternatively spliced forms), and naturally occurring allelic variants of BACE2. The identifier of the human BACE2 polypeptide in the swissprot database is Q9Y5Z0 (Seq. Id. No. 3).

The term “BACE2 variant” refers to amino acid sequence variants of a native sequence BACE2, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence BACE2.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “Tmem27 derived peptide” refers to a peptide having at least 80% sequence identity with a contiguous stretch of at least 5 amino acids of a Tmem27 polypeptide and said peptide being cleavable by BACE2.

The term “Angiotensin converting enzyme 2 (ACE2)” is used herein to refer to native ACE2 sequence from any animal, e.g. mammalian, species, including humans, and ACE2 variants. The ACE2 polypeptides may be isolated from a variety of sources, including human tissue types or prepared by recombinant and/or synthetic methods.

“Native sequence ACE2” refers to a polypeptide having the same amino acid sequence as a ACE2 polypeptide occurring in nature regardless of its mode of preparation. A native sequence ACE2 may be isolated from nature, or prepared by recombinant and/or synthetic methods. The term “native sequence ACE2” specifically encompasses naturally occurring truncated or secreted forms, naturally occurring variant forms (e.g. alternatively spliced forms), and naturally occurring allelic variants of ACE2. The identifier of the human ACE2 polypeptide in the swissprot database is Q9BYF1 (Seq. Id. No. 4).

The term “ACE2 variant” refers to amino acid sequence variants of a native sequence ACE2 containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence ACE2. The term “ACE2 variant” refers as well to ACE2 fragments which can be processed by BACE2 e.g. truncated ACE2 polypeptides which are still a substrate for BACE2.

The term “ACE2 derived peptide” refers to a peptide having at least 80% sequence identity with a contiguous stretch of at least 5 amino acids of a ACE2 polypeptide and said peptide being cleavable by BACE2.

The compounds identified by the inventive assays can be used for the development of medicaments for the treatment of a metabolic disorder, preferably type 2 diabetes.

Fluorophore fluorescence resonance energy transfer (FRET) assays which can be used in a method of the present invention to identify a BACE2 inhibitor are well know to a person skilled in the art. A suitable FRET assay is e.g. described in Gruninger-Leitch et al. [Substrate and inhibitor profile of BACE (beta-secretase) and comparison with other mammalian aspartic proteases. Journal of Biological Chemistry (2002) 277(7) 4687-93]. In summary, a peptide is designed that is cleaved by the protease. The peptide is labelled with e.g. dabsyl at the N terminus and e.g. Lucifer Yellow at the C-terminus, such that for an intact peptide the Lucifer Yellow fluorescence is quenched by the dabsyl. When the peptide is cut by BACE2, the quenching is removed and a fluorescent signal is generated.

Fluoroscence quench assays which can be used in a method of the present invention to identify a BACE2 inhibitor are well known to a person skilled in the art. A suitable assay is e.g. described in Marine N. et al. [Angew. Chem. Int. Ed. 2004, 43, 3798-3801].

### SHORT DESCRIPTION OF THE FIGURES

FIG. 1 shows the results of Western blot analysis showing preservation of full-length TMEM27 in a cellular assay (INS-TMEM27/BACE2 cell line) in a dose-dependent manner by (R)-2-Amino-6-[2-(3′-methoxy-biphenyl-3-yl)-ethyl]-3,6-dimethyl-5,6-dihydro-3H-pyrimidin-4-one (Inhibitor),

FIG. 2 shows TMEM27 ELISA readings from culture supernatants of INS1e cells stably expressing human TMEM27 and treated with a BACE2 inhibitor,

FIG. 3 shows the results of Western blot analysis showing preservation of full-length TMEM27 by (R)-2-Amino-6-[2-(3′-methoxy-biphenyl-3-yl)-ethyl]-3,6-dimethyl-5,6-dihydro-3H-pyrimidin-4-one and concomitant inhibition of shedding into the culture supernatant. Obtained in HEK293 cells stably transfected with TMEM27 and BACE2 under CMV promoter control,

FIG. 4 shows the results of a cellular assay. Human pancreatic islets treated with BACE2 inhibitor (R)-2-Amino-6-[2-(3′-methoxy-biphenyl-3-yl)-ethyl]-3,6-dimethyl-5,6-dihydro-3H-pyrimidin-4-one show preservation of full-length TMEM27. ˜2000 islets (male; BMI=23.4; 19 y/o) incubated 48 hours with and without 10 μM inhibitor,

FIG. 5a shows the results of a cellular assay for BACE2 inhibitors by measuring changes in proliferation rates of Min6 cells,

FIG. 5b shows Western blots of cells used in assay of FIG. 5a,

FIG. 6 shows the results of a cellular assay for BACE2 inhibitors by measuring changes in proliferation rates of Ins1e cells,

FIG. 7 shows FRET assay results with peptides derived from APP, TMEM27 and unrelated proteins,

FIG. 8 shows a FRET assay dose response curve with TMEM27 peptide and a reference BACE2 inhibitor and

FIG. 9 shows activity of BACE2 in a FRET assay on peptide substrates derived from relevant peptides.

### EXPERIMENTAL PART

### Assay for BACE2 Inhibition by Measuring Cellular TMEM27 Cleavage

1. Stable Cell Line:

INS-TMEM27/BACE2 represents a stable cell line allowing inducible expression (using the TetOn system) of both hTMEM27 and hBACE2 in a doxycycline-dependent manner, which was established by three-steps of consecutive stable selections with, respectively, neomycin (G418), hygromycin, and Zeocin.

2. Culture, Passage, and Storage:

a). Culture

Basic INS-1 cell culture medium:

| | |
|---|---|
| RPMI1640 + GlutamaxI, 61870-010 | 500 ml |
| Strep-Pen 100X | 5 ml |
| FCS | 50 ml |
| 100 mM pyruvate + 5 mM BMCTE | 5 ml |
| (100 ml (100 mM) pyruvate + 50 ul | |
| beta-mercaptoethanol) | |
| 1M HEPES | 5 ml |
| | Basic INS-1 medium |

Complete medium: INS-TMEM27/BACE2 triple stable cell line is cultured in basic INS-1 medium containing selection pressures: 100 μg/ml G418; 100 μg/ml hygromycin, 250 μg/ml zeocin.
Change the cell media twice a week

b). Passage:

Trypsinize cells once a week: rinse cells once with PBS, incubate with 2 ml trypsin for 3 min at room temperature, add 10 ml complete culture medium, then split 1 to 3.

c) Storage:
Resuspend ˜10×106 cells in 1 ml of FCS containing 10% DMSO
Place them in a freezing capsule
Place cells at −80° C. overnight
Store cells in liquid N2

3. Assay:

a). Induction of hTMEM27 Expression and its Cleavage by BACE2:
Seed INS-TMEM27/BACE2 cells in 96-well plates.
2-3 days after culture in complete medium, add doxycycline to a final concentration of 500 ng/ml. (Doxycycline should be prepared freshly and dissolved in H2O.)

b). Measurement of BACE2 Inhibition.
Add desired concentrations of BACE2 compounds 2 hours pre-administration of doxycycline.
Incubate for further 46 hours.
Detect the shed hTMEM27 in the culture medium using Western blot or ELISA or measure full length TMEM27 in cell lysate by Western blot.

4. Results with a Western Blot Readout

FIG. 1: Western blot showing preservation of full-length TMEM27 in the cellular assay in a dose-dependent manner by the BACE2 inhibitor (R)-2-Amino-6-[2-(3′-methoxy-biphenyl-3-yl)-ethyl]-3,6-dimethyl-5,6-dihydro-3H-pyrimidin-4-one.

5. Results with an ELISA Readout

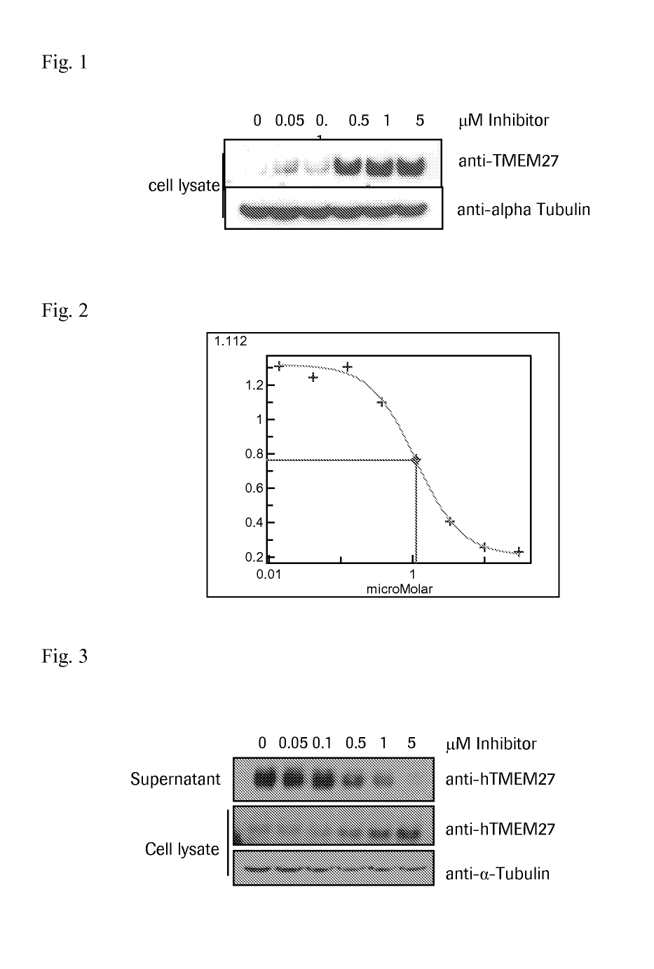

FIG. 2: The chart shows TMEM27 ELISA readings from culture supernatants of INS1e cells stably expressing human TMEM27 and treated with a BACE2 inhibitor as described in the protocols. Shed TMEM27 is reduced with higher concentrations on BACE2 inhibitor and an IC50 for the inhibitor can be deduced with standard curve-fitting programs, such as Xlfit. In this case an IC50 of 1.112 microMolar is determined.

6. Alternative Cellular System

FIG. 3: Similar results are obtained in HEK293 cells stably transfected with TMEM27 and BACE2 under CMV promoter control. The Western blot shows preservation of full-length TMEM27 by (R)-2-Amino-6-[2-(3′-methoxy-biphenyl-3-yl)-ethyl]-3,6-dimethyl-5,6-dihydro-3H-pyrimidin-4-one and concomitant inhibition of shedding into the culture supernatant.

7. Detection of BACE2 Inhibition by Measuring TMEM27 Cleavage in Isolated Human Pancreatic Islets.

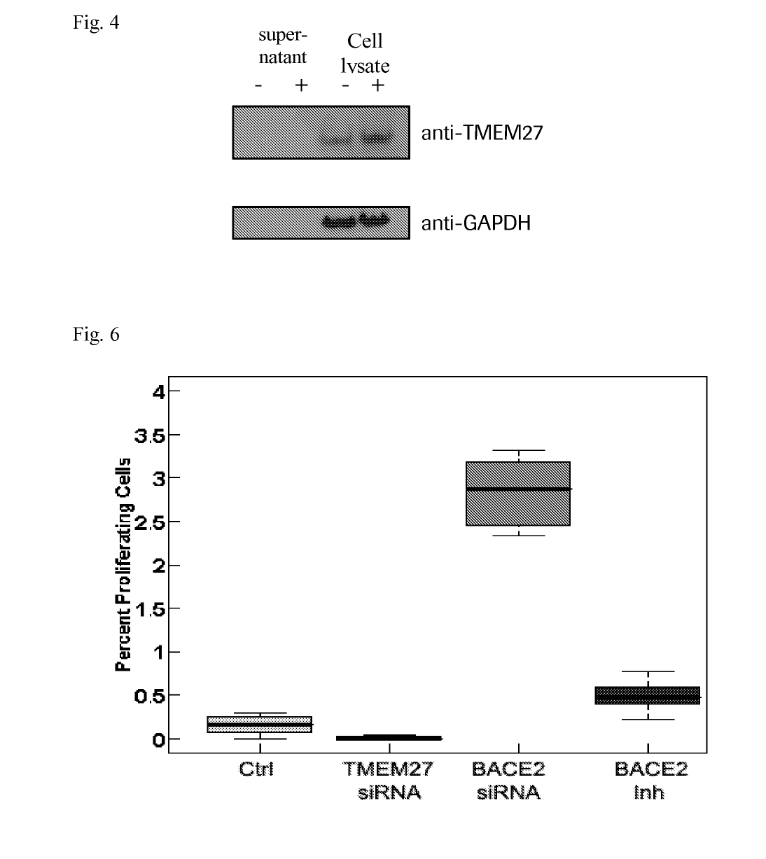

FIG. 4: Human islets treated with BACE2 inhibitor (R)-2-Amino-6-[2-(3′-methoxy-biphenyl-3-yl)-ethyl]-3,6-dimethyl-5,6-dihydro-3H-pyrimidin-4-one show preservation of full-length TMEM27. ˜2000 islets (male; BMI=23.4; 19 y/o) incubated 48 hours with and without 1 μM inhibitor. TMEM27 is not detected in supernatant, but effect on lysed cells is apparent.

Assay for BACE2 Inhibitors by Measuring Changes in Proliferation Rates of Min6 Cells

1. Cell Culture, Passage, and Storage

a). Culture

MIN6 B1 or INS-1e were respectively cultivated in standard MIN6 culture medium as described in the following table or in basic INS-1 medium as described earlier.

Standard complete MIN6 cell culture medium:

| | |
|---|---|
| D-MEM high glucose + glutamine + | 500 ml |
| pyruvate (Invitrogen #61870) | |
| Strep-Pen 100X (10000 U/m1) | 5 ml |
| FCS | 75 ml |
| 5 mM BMCTE | 5 ml |
| (100 ml D-MEM + 50 ul beta- | |
| mercaptoethanol) | |
| | Complete MIN6 medium |

Media was changed twice a week

b). Passage:

Trypsinize cells once a week: rinse cells once with PBS, incubate with 2 ml trypsin for 3 min at room temperature, add 10 ml complete culture medium, then split 1 to 3.

If desired, after having resuspended the cells in 10 ml complete culture medium, cells were counted using a Neubauer chamber and diluted to the wished cell density.

c) Storage:
Resuspend ˜10×106 cells in 1 ml of FCS containing 10% DMSO
Place them in a freezing capsule
Place cells at −80° C. overnight
Store cells in liquid N2

2. Cell Preparation Protocol

50′000 MING B1 cells per well were seeded in a 96-well plate using standard trypsinization method. The following day, serum-supplemented complete medium (described earlier) was removed and replaced by serum-free and low glucose medium (Invitrogen) supplemented with the compound as follows: 10 nM exendin-4 (Sigma-Aldrich) as a positive control was added to the cells every 12 h, to avoid degradation. 1 μM RO519996 was added at the start of the experiment. As an absolute positive control cells were also grown in FCS supplemented standard media. Cells were incubated for 48 hours.

After this incubation, medium was discarded and replaced by the serum-supplemented medium containing 10 nM EdU (Invitrogen) and incubated for 1 h at 37° C. Supernatants were then carefully removed and cells fixed, permeabilised and stained with the appropriate dyes according to the manufacturer's protocol (Invitrogen, Click-It EdU kit).

3. Proliferation Assay: Image Acquisition, Microscopy

Spinning-disc confocal fluorescence microscopy of the 96-well plate has been performed on the high-throughput automated imaging system Opera™ QEHS (PerkinElmer Cellular Technologies, Hamburg, Germany). The nuclear stain (Hoechst or DAPI) was excited by the 405 nm line of a solid state laser or by a mercury UV lamp in wide-field illumination. The Alexa Fluor 488 labeled proliferation marker was excited by a 488 nm solid state laser. The excitation intensity and duration of all illumination sources was adjusted in each experiment to account for differences in labeling efficiencies, to optimize brightness and contrast, and to minimize bleaching (typically, 50 mW laser output, 40-400 ms integration time). In each well, typically 12 pairs of scanning images were recorded through a UAPO 20×NA 0.7 water immersion objective lens (Olympus) and optimized filter sets by 2 independent high quantum-efficiency 12 bit CCD cameras (1.3 mega pixels monochrome). Any residual illumination heterogeneity, image shift or distortion is corrected for using separately acquired images from calibration samples.

4. Proliferation Assay: Image Analysis

Confocal micrographs were analyzed using the proprietary software environment of PerkinElmer's Opera (Acapella 1.8, PerkinElmer Cellular Technologies, Hamburg, Germany). First, the location of each cell is identified by segmentation of the nuclear stain image. Once the nuclei have been localized and their outline and area has been determined, the intensity of the EdU stain image at the location of the nucleus is quantified for each cell. This way, any unspecific EdU stain can be disregarded. After application of a suitable threshold, the proliferation rate is calculated as the number of EdU-positive cells divided by the total number of identified cells, and is thus independent of the laser intensity, the labeling efficiency, or the fluorophore brightness.

5. Proliferation Assay: Statistical Analysis

The experimentally determined proliferation rate is calculated for different treatment conditions, including a positive control (high FCS concentration) and a negative control (starved cells). Significance of differences between conditions has been tested against the Null hypothesis H0, that two conditions have identical proliferation rate, using a two-sided t-test of the arcsine-transformed proliferation rates with Bonferroni-correction for multiple testing and was quantified by the corresponding p-values.

6. Results: Proliferation Assay

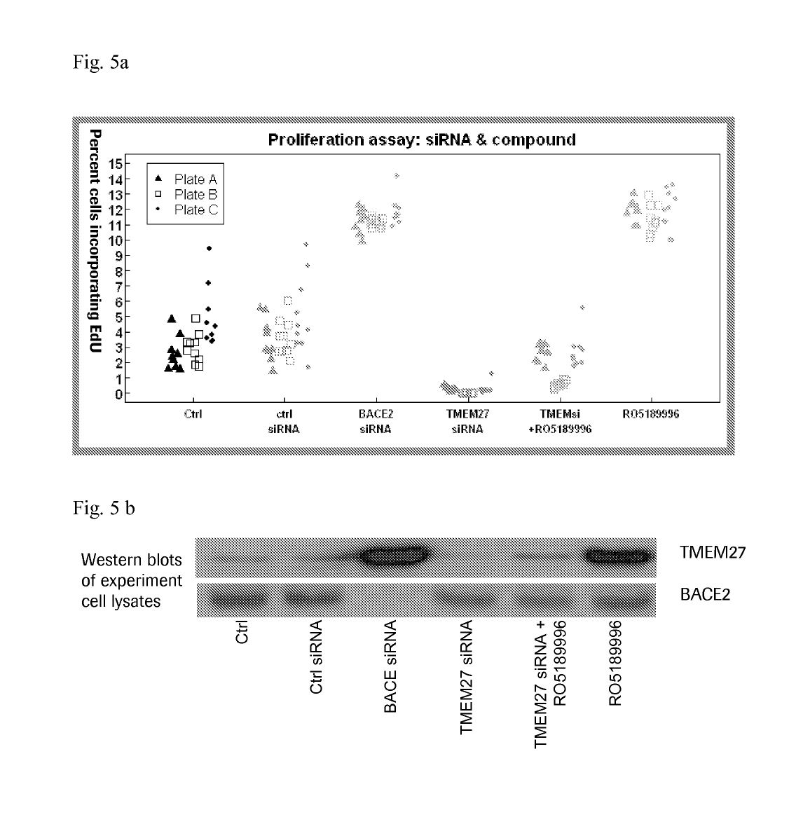

Compared to the basal proliferation rate of starved cells, all three tested conditions, i.e. FCS, Exendin-4, and (R)-2-Amino-6-[2-(3′-methoxy-biphenyl-3-yl)-ethyl]-3,6-dimethyl-5,6-dihydro-3H-pyrimidin-4-one (FIG. 5a), exhibit a highly significant proliferation increase (p<10-6). Moreover, the Roche compound significantly exceeds even Exendin-4 in proliferation enhancement. The two different preparations (plate A, plate B) showed no effect on proliferation (p=0.21), demonstrating the reproducibility of the assay.

FIG. 5a: Induction of Min6 cell proliferation. Data are from three independent preparations of starved Min6 cells transformed with BACE2, TMEM27 or control siRNA to adjust the TMEM27 concentrations. Proliferation is strongly correlated with TMEM27 levels. The assay can also be used as a readout of BACE2 activity with proliferation correlated to the level of BACE2 inhibition.

FIG. 5b shows Western blots of cells used in assay of FIG. 5a.

7. Alternative Cell Systems: Ins-1e Cells

The Insle assay was performed with the same protocol as for Min6 cells, except that standard Insle culture medium was used for cell culture. Proliferation was measured by incubation with 10 μM BrdU for 30 minutes and staining of cells with Alexa488-anti-BrdU (Invitrogen) antibody according to the manufacturer's protocols.

FIG. 6: Demonstration of Insle proliferation induced by inhibition of BACE2. Cells treated with a BACE2 inhibitor (BACE2 Inh) have a significantly higher (p<0.05) proliferation rate than control cells with basal proliferation rates after 2 days growth on low-glucose medium.

Assay for BACE2 Inhibitors by Measuring Cleavage of TMEM27- and ACE2-Derived Peptides

1. Description of FRET Assay

The FRET assay was performed essentially as described in Grüninger-Leitch et al. [Substrate and inhibitor profile of BACE (beta-secretase) and comparison with other mammalian aspartic proteases. Journal of Biological Chemistry (2002) 277(7) 4687-93]. In summary, a peptide is designed that is cleaved by the protease. The peptide is labelled with dabsyl at the N terminus and Lucifer Yellow at the C-terminus, such that for an intact peptide the Lucifer Yellow fluorescence is quenched by the dabsyl. When the peptide is cut by BACE2, the quenching is removed and a fluorescent signal is generated.

The assay was performed as described in Grueninger et al. 2002 at pH 4.5 using a substrate concentration of 5 μM. All FRET-peptides had the format described.

A FRET peptide based on the TMEM27 sequence was devised. dabsyl—QTLEFLKIPS—LucY (Seq. Id. No. 1). BACE2 had a high activity against this sequence, which is unrelated to the known APP-based substrates. Conversely, BACE1 had insignificant activity against this peptide.

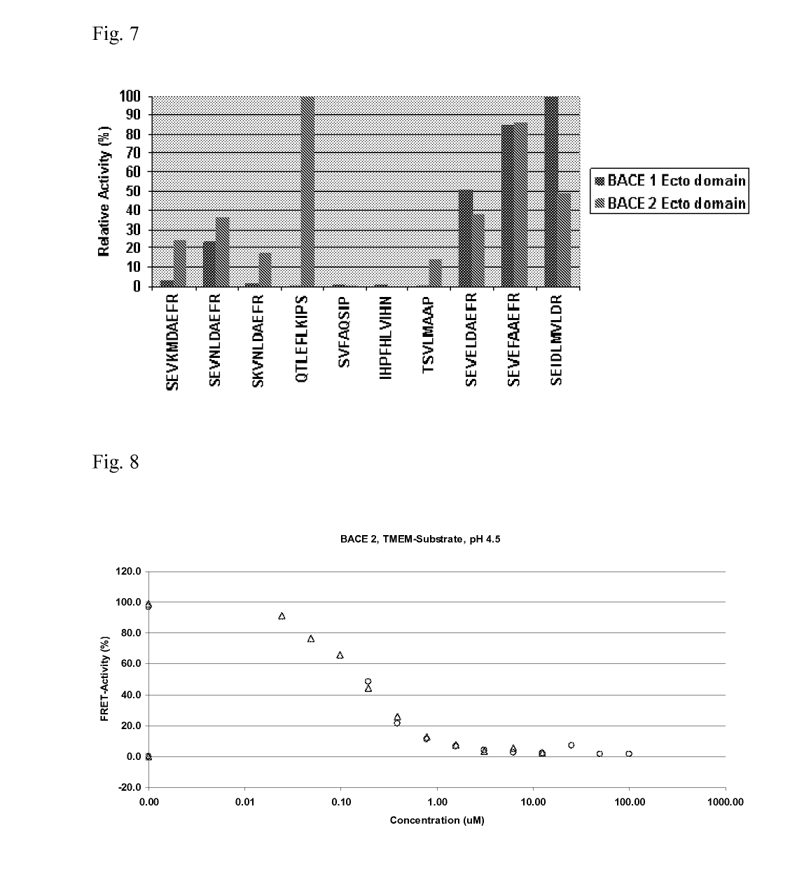

FIG. 7: Comparison of BACE1 and BACE2 activity against different FRET peptides. FRET assay results with dabsyl and Lucifer yellow labelled peptides derived from APP, TMEM27 and unrelated proteins. The peptides used in the assay have the following amino acid sequences:

| Amino acid | Seq. Id. |
|---|---|
| sequence | No. |
| SEVKMDAEFR | 16 |
| SEVNLDAEFR | 17 |
| SKVNLDAEFR | 19 |
| QTLEFLKIPS | 1 |
| SVFAQSIP | 20 |
| IHPFHLVIHN | 21 |
| TSVLMAAP | 22 |
| SEVELDAEFR | 23 |
| SEVEFAAEFR | 24 |
| SEIDLMVLDR | 25 |

FIG. 8: FRET assay dose response curve with dabsyl and Lucifer yellow labelled TMEM27 peptide (Seq. Id. No. 1) and a reference BACE2 inhibitor. BACE2 inhibition is measured in the same assay. In this example, BACE2 is inhibited by addition of varying concentrations of (R)-2-Amino-6-[2-(3′-methoxy-biphenyl-3-yl)-ethyl]-3,6-dimethyl-5,6-dihydro-3H-pyrimidin-4-one to the assay. FRET activity is normalised to the maximum obtained without inhibitor. Giving an IC50 for this compound of 0.084 μM as calculated by the Excel XLFit add-in.

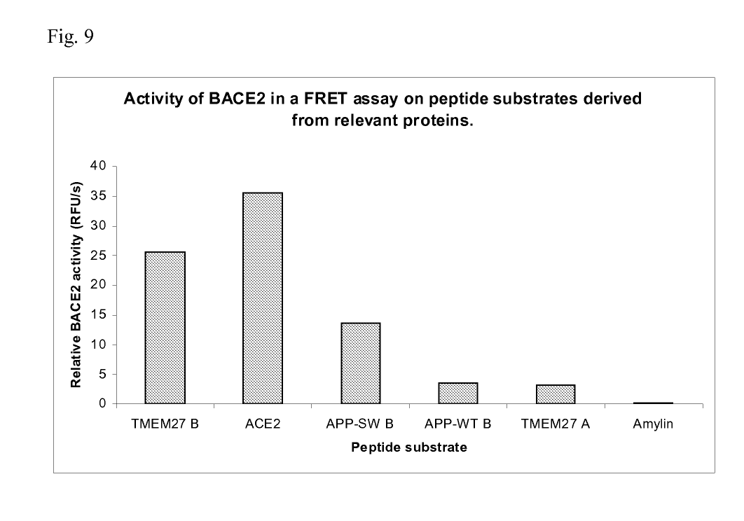

FIG. 9: A peptide was also devised based on the sequence of ACE2 at the site equivalent to the BACE2 cleavage site in TMEM27 and with the sequence dabsyl-NSLEFLGIQP-lucifer yellow (Seq. Id. No. 14). This substrate was cleaved by BACE2 with an efficiency greater than that of the TMEM27 substrate. Alternative substrates derived from different regions of TMEM27, APP and from amylin were cleaved less efficiently. The peptides used in the assay have the following amino acid sequences:

| | Amino acid | Seq. Id. |
|---|---|---|
| Peptide | sequence | No. |
| TMEM 27 B | QTLEFLKIPS | 1 |
| ACE 2 | NSLEFLGIQP | 14 |
| APP SW | SEVNLDAEFR | 15 |
| APP WT | SEVKMDAEFR | 16 |
| TMEM 27 A | RINNAFFLND | 17 |
| Amylin | GSNTYGKRNA | 18 |

2. Description of MR121 Assay

In a related assay, a peptide was devised with the same sequence, which was labeled at the N-terminus with MR121 and at the C-terminus with Tryptophan.

Substrate peptides derived from TMEM27 with activity in the MR121-peptide cleavage assay.

| Amino acid | Relative | Seq. Id. |
|---|---|---|
| sequence | activity | No. |
| 1. C QTLEF W | 21 | 5 |
| 2. C QTLEFL W | 32 | 6 |
| 3. C QTLEFLK W | 62 | 7 |
| 4. C QTLEFLKI W | 96 | 8 |
| 5. C QTLEFLKIP W | 75 | 9 |
| 6. C QTLEFLKIPS W | 91 | 10 |
| 7. C LEFLKI W | 24 | 11 |
| 8. C TLEFLKI W | 28 | 12 |
| 9. C QELEFLKI W | 100 | 13 |

Preparation of BACE2 for Assays

BACE2 enzyme ectodomain was prepared as described in Ostermann et al [“Crystal Structure of Human BACE2 in Complex with a Hydroxyethylamine Transition-state Inhibitor.” Journal of Molecular Biology 355 (2006) 249-261].

Preparation of Expression System

DNA sequence corresponding to amino acids 20-465 in the BACE2 translated amino acid sequence was cloned into the NdeI and XhoI sites of pET17b such that the BACE2 sequence was in frame with the 5′ ATG and was terminated by a stop codon. The plasmid is termed pET17b-BACE2(ecd)

BL21(DE3; pLysS) cells were transformed with pET17b-BACE2(ecd) to give an IPTG-inducible E. coli expression system.

These cells were grown in standard LB expression medium supplemented with Ampicillin and chloramphenicol to an OD600˜0.5. IPTG is added to a final concentration of 1 mM. Incubation is continued for 3 hours at 37° C. Cells are harvested by centrifugation at 3000 g for 10 minutes

IB Preparation
Mechanical disruption of cells by sonication or using a Constant Systems cell disrupter.
Harvest inclusion bodies by centrifugation at 5000 g 10 minutes
Wash pellet 4 times in 50 mM Tris, pH 8.0
Resuspend pellet in minimum volume of denaturation buffer.
Refolding
Dilute dissolved protein to between 10 and 21 mg/ml in denaturation buffer.
Dilute denatured protein by rapid dilution into 20× volume refolding buffer I.
Incubate with gentle stirring overnight.
Dilute by rapid dilution into 20× volume of refolding buffer II.
Incubate with gentle stirring for 2-5 days.

Purification

Add 300 ml saturated ammonium sulphate (˜4M) per litre refolded protein solution.

Apply to Amersham HiPrep butyl sepharose 6 FF HIC column, pre-equilibrated with HIC buffer B. Wash with 3 CV HIC buffer B, then elute with buffer A applied in a gradient.

Most active fractions pooled and dialysed against >20× volume 10 mM Tris, pH8.0; 150 mM

NaCl.

Buffers:

Denaturation buffer: 50 mM Tris, pH 8.0, 8.0M GuHCL, 30 mM dithioethyritol

Refolding buffer I: 3M GuHCl, 0.7M Arginine base, 0.5 mM GSSG, 1.0 mM GSH pH 10.4 with HCl

Refolding buffer II: 1M NaCl, 0.7M Arginine base, 0.5 mM GSSG, 1.0 mM GSH pH 9.4 with HCl

HIC buffer A: 10 mM Tris, pH8.0

HIC buffer B: 10 mM Tris, pH 8.0, 1M NaCl, 1.5M AmSO₄

Assay buffer: 10 mM Tris, pH 8.0, 150 mM NaCl

Description of Antibodies Used and their Generation.

1. Whole Cell Immunization for Production of 9/24 Antibody

Immunization of Swiss albino mice was performed with INS-1e cells stably expressing hTMEM27 by repeated injection of living cells. As soon as the animals showed a specific immune response to hTMEM27, the spleen cells were removed and fused to Ag8 cells according to G. Köhler and C. Milstein (1975) “Continuous cultures of fused cells secreting antibody of predefined specificity”. Nature 256:495-497.

2. Protein Immunization for Production of 3/3 and 1/33 Antibody

Some monoclonal antibodies where obtained by subcutaneous immunization of the animals in 2-3 weeks intervals with purified hTMEM proteins (produced in E. coli), followed by fusion of the spleen cells as described.

3. Use of Antibodies 9/24 antibody was used exclusively for the ELISA. 1/33 for western blots where the shed protein is detected and the ELISA. 3/3 for Western blots where the full length protein is detected. 1/33 and 3/3 antibodies were conjugated to horseradish peroxidise using standard methods as required by the protocols.

Description of an ELISA Assay for Soluble TMEM27

1. Assay Protocol

In standard 96-well immunoassay plates

COATING: TMEM27-9/24 antibody, 5 μg/ml in PBS, 100 μl/well, over night at 40 C

WASH: 2 times with PBS-Tween

BLOCKING: B-Buffer, 200 μl/well, 1 h at 370 C

WASH: 2 times with PBS-Tween

SAMPLES: culture supernatants, dilutions in B-Buffer, 54 μl/well, 1 h at 370 C

WASH: 4 times with PBS-Tween

CONJUGATE: Ec-1/33-HRPO, 1 μg/ml in B-Buffer, 54 μl/well, 1 h at room temperature

WASH: 4 times with PBS-Tween

SUBSTRATE: 100 μl/well, 5 min, stop of the substrate reaction with 100 μl/well of 1M sulfuric acid

READ OUT: OD measurement at 450 nm

While there are shown and described presently preferred embodiments of the invention, it is to be distinctly understood that the invention is not limited thereto but may be otherwise variously embodied and practiced within the scope of the following claims.

1. A method for identifying a BACE2 inhibitor comprising:
providing a beta cell expressing a Tmem27 polypeptide wherein proliferation of the cell is dependent on BACE2 mediated Tmem27 cleavage, contacting a mixture comprising a BACE2 polypeptide and the cell expressing the Tmem27 polypeptide with a candidate compound and determining whether the candidate compound modulates cell proliferation, wherein an increase of cell proliferation is indicative for a BACE2 inhibitor.

2. The method of claim 1, wherein the cell expresses Tmem27 polypeptide and BACE2.

3. The method of claim 2, wherein the beta cell line, is selected from the group consisting of a MIN6 B1 or INS-le cell line.

4. (canceled)

5. (canceled)

6. A method for identifying a BACE2 inhibitor comprising:
contacting BACE2 and a peptide comprising a Tmem27 derived BACE2 cleavage site or an ACE2 derived BACE2 cleavage site with a candidate compound and determining cleavage of the peptide, wherein cleavage of the peptide is indicative for a BACE 2 inhibitor.

7. The method of claim 6, wherein the Tmem27 derived peptide or the ACE2 derived peptide comprises a peptide having the sequence set forth in Seq. Id. No. 1 or set forth in Seq. Id. No. 12.

8. The method of claim 7, wherein cleavage of the Tmem27 derived peptide or the ACE2 derived peptide is determined in a FRET assay.

9. The method of claim 8, wherein the Tmem27 derived peptide or the ACE2 derived peptide is labeled with a dabsyl at the N-terminus and a fluorescent dye at the C-terminus.

10. The method of claim 9, wherein the fluorescent dye is Lucifer yellow.

11. The method of claim 6, wherein cleavage of the Tmem27 derived peptide or the ACE2 derived peptide is determined in a fluorescence quench assay.

12. The method of claim 11, wherein the Tmem27 derived peptide or the ACE2 derived peptide is labeled with MR121 at the N-terminus and tryptophan at the C-terminus.

13. (canceled)

14. (canceled)
